(19) United States
(12) Patent Application Publication
(10) Pub. No.: US 2015/0132738 A1
(43) Pub. Date: May 14, 2015

(54) Method For Identification Of Non-Hematogeneous Karocytes Enriched From Body Fluid Of Humans Or Animals

(71) Applicant: Yanping Li, Haidian District Beijing (CN)

(72) Inventor: Yanping Li, Haidian District Beijing (CN)

(21) Appl. No.: 14/355,671

(22) PCT Filed: Sep. 7, 2012
(86) PCT No.: PCT/CN2012/081147

§ 371 Date: Jun. 20, 2014

(30) Foreign Application Priority Data
Nov. 1, 2011 (CN) 201110340544.9

(51) Int. Cl. G01N 33/574; C12Q 1/68

(52) U.S. Cl. CPC G01N 33/57492; C12Q 1/6886; C12Q 2600/156
USPC 435/2; 435/6.11; 435/7.23

(57) ABSTRACT

Disclosed is a method for identification of non-hematogenous karyocytes enriched from body fluid of humans or animals, wherein immunofluorescent staining of antibodies against human leukocytes and fluorescence in situ hybridizationof chromosome are carried out synchronously to distinguish hematogenous karyocytes and the interference produced by the hematogenous karyocytes during the judgement on tumour cells is removed effectively, thus achieving efficient and rapid enrichment and extraction from human body fluid of non-hematogenous rare cancer cells originating from the epithelium or not originating from the epithelium and effective identification. The method provided by the present invention does not need to lyse and break red blood cell and without influencing the targeted cell in human body fluid, thus the original cell shape is maintained very well, which is convenient for the subsequent identification and judgement of cells.

### TECHNICAL FIELD OF THE INVENTION

The present invention mainly relates to a method of using synchronous immunofluorescent staining and fluorescent in situ hybridization technology for identification of malignant epithelium originated (solid tumour) or non-epithelium originated (melanoma) tumour cell enriched from body fluid of human or animal.

### BACKGROUND OF THE INVENTION

The huge practical and potential clinical significance of extraction and detection of malignant epithelial cell (tumour cell) in body fluid (hydrothorax or pleural effusion, abdominal dropsy or ascites, cerebrospinal fluid, lymph and/or blood etc.) of human have already been verified by people. Many collected human body fluid samples are often mixed with a great number of red blood cells and leukocytes, which causes great interference to the judgement of experiment operations and results. Currently, a common way to remove red blood cell during clinical or medical experiments is to lyse and remove red blood cell through hypotonic lysis. However, a certain degree of damage is caused to many malignant epithelial cell (tumour cell) during hypotonic lysis along with cracking of the red blood cell, thus changing the cell shape and characteristics to further influence correct judgment of such cell.

It's been widely reported that Fluorescence in situ Hybridization (FISH) technology is applied in detection of tumour cell in pathological tissue. One of the major technical applications is identification of chromosome number (normal cell chromosome are diploid) of interphase tumour cell in body fluid by using a fluorescence-labelled chromosome centromeric probe, so as to discover non-diploid chromosome in the tumour cell, i.e. hyperdiploid chromosome or polyploidy chromosome, thereby identifying the tumour cell. The FISH method has been widely used in diagnosis of blood tumour before, but is rarely reported to have been applied in detection of solid tumour cell (non-blood tumour) in body fluid including blood. The most common problem of using the FISH method for detection of tumour cell in blood samples is how to distinguish hematogenous cell which have abnormal chromosome number, such cell may be seen in thrombocythemia-derived numerical abnormalitie of chromosome of some leukocytes in blood, or natural numerical abnormalitie of chromosome of a small number of leukocytes in human bodies, or chromosome probe non-specific staining-induced numerical abnormalitie of false positive chromosome of blood cell. Therefore, it is very important to distinguish hematogenous cell and non-hematogenous cell effectively during identification and judgement of non-hematogenous solid tumour cell through FISH. However, there have been no reports about researches in this aspect.

Besides, although detection of circulating tumour cell in blood by using a non-synchronous FISH method based on tumour epithelial cell staining has been reported by a few articles, i.e. FISH and staining of anti-tumour cell marker antibody are carried out alternately and non-synchronously, no clinical use value is exhibited because a great number of detached solid tumour cells have lost the characteristics of tumour epithelial cell in a liquid environment due to the complete change of the growth environment, and the loss of the characteristics directly affect the antibody immune staining of the tumour cells, resulting in a great number of false negatives.

Due to the reasons above, the present invention uses a centrifugation method based on a composition of a density separating agent (i.e. cell separation medium) for rapid and efficient separation of red blood cell from malignant epithelial cell and other karyocyte in body fluid of human, Surprising good experiment results are achieved without an experiment step of breaking and lysing the red blood cell, which proves that effective separation of most red blood cells and plasma proteins in samples of body fluid of human from other karyocyte including tumour cell in the body fluid specimen, can be implemented rapidly and efficiently by using the composition without influencing the targeted cell (e.g. tumour cell in the body fluid) in the human body fluid, thus the original cell shape is maintained very well, which is extremely convenient for the subsequent identification and judgement of cells. After removing leukocyte with immunomagnetic beads, malignant non-hematogenous epithelial cell (tumour cell), vascular endothelial cell and so on in the body fluid can be rapidly enriched and extracted. During subsequent identification of malignant non-hematogenous epithelial cell with the FISH method, immunofluorescent staining of antibody against human leukocyte surface antigen is carried out synchronously to eliminate interference of any hematogenous cell, At the same time, tumour marking (e.g. Her2, CEA, CA19-9 or stem cell marker CD133) or immunofluorescent staining of epithelial cell marker (e.g. cytokeratin) may be carried out synchronously on the same specimen, so rapid and accurate identification of highly-sensitive and highly-specific characteristics of malignant non-hematogenous tumour cell enriched from sample of the body fluid of humans is achieved.

### SUMMARY OF THE INVENTION

The present invention relates to a method for identification of malignant non-hematogenous karyocyte enriched from body fluid of human or animal, wherein immunofluorescent staining of antibody against human or animal leukocyte and FISH of chromosome are carried out synchronously to distinguish hematogenous karyocyte. The malignant non-hematogenous karyocyte can be tumour cell or vascular epithelial cell, for example.

The antibody against human or animal leukocyte in the immunofluorescent staining specifically identify antigen of one or more of the following surface markers of the human leukocyte: CD3, CD31, CD34, CD45, CD50, CD69, CD84 or CD102.

A fluorescence labelling-probe or a probe combined group for the FISH of chromosome can specifically identify any one or more human or animal chromosomes.

The present invention further relates to a method for rapid and efficient separation of red blood cell from such non-hematogenous karyocyte as tumour cell and vascular epithelial cell etc. from body fluid of human, wherein the method does not need a step of breaking and lysing red blood cell, wherein a cell separation medium is applied to separation, the specific gravity of the cell separation medium at 20□ is 1.07156 to 1.07738 gr/ml; the cell separation medium includes any one or two or more components selected from the following reagents: polyvinylpyrrolidone-coated colloidal silica; polysaccharide; sodium diatrizoate or a derivative thereof; a nonionic polymer consisting of sucrose and epichlorohydrin; or any sugar-containing solution, e.g. dextran or sucrose; an iodinated small molecule compound, e.g. metrizamide, or any protein solution. The use of the new cell separation medium of the present invention may greatly shorten the centrifugation time, and stably improve the cell recovery rate from the original 60±20% to 95%-100%.

The present invention further relates to a method for enrichment of malignant non-hematogenous karyocyte from body fluid of human, wherein the method does not need a step of breaking and lysing red blood cell, and the method includes: performing centrifugation to remove plasma proteins; placing the centrifuged supernatant at the top layer of a cell separation medium and performing centrifugation over again; taking the supernatant and adding immunomagnetic beads against coupling of leukocyte to perform incubation and then adsorbing the immunomagnetic beads; performing centrifugation to enrich cell precipitate.

The present invention further relates to a method for enrichment of non-hematogenous karyocyte from body fluid of human, wherein the method does not need a step of breaking and lysing red blood cell, and the method includes: performing centrifugation to collect or remove plasma protein; placing the centrifuged supernatant at the top layer of a cell separation medium and performing centrifugation over again; taking the supernatant and add immunomagnetic beads against coupling of leukocyte antibody to perform incubation and then adsorbing the immunomagnetic beads; placing the supernatant at the top layer of the cell separation medium and performing centrifugation over again; performing centrifugation for the supernatant to enrich cell precipitate.

The present invention further relates to a method for enrichment and identification of non-hematogenous karyocyte from body fluid of human or animal, and the method includes: performing centrifugation to remove plasma protein; placing the centrifuged supernatant at the top layer of a cell separation medium and performing centrifugation over again; taking the supernatant and adding immunomagnetic beads against coupling of leukocyte antibody to perform incubation and then adsorbing the immunomagnetic beads; performing centrifugation, taking enriched cell precipitate, wherein immunofluorescent staining of antibody against human or animal leukocyte and FISH of chromosome are carried out synchronously to the enriched cell precipitate to distinguish hematogenous karyocyte.

The present invention further relates to a method for enrichment and identification of non-hematogenous karyocyte from the body fluid of human or animal, and the method includes: performing centrifugation to remove plasma protein; placing the centrifuged supernatant at the top layer of a cell separation medium and performing centrifugation over again; taking the supernatant and adding immunomagnetic beads against coupling of leukocyte antibody to perform incubation and then adsorb the immunomagnetic beads; then placing the supernatant at the top layer of a cell separation medium and performing centrifugation over again; taking and performing centrifugation for the supernatant and taking enriched cell precipitate, wherein immunofluorescent staining of antibody against human or animal leukocyte and FISH of marker against human or animal tumour cell or epithelial cell marker and chromosome are carried out synchronously to the enriched cell precipitate to distinguish non-hematogenous tumour cell.

The body fluid of human or animal includes, but is not limited by hydrothorax or pleural effusion, abdominal dropsy or ascites, cerebrospinal fluid, lymph and/or blood, urine, sputum, lavage fluid and so on. The non-hematogenous karyocyte in the body fluid of human or animal are selected from a group consisting of the followings: any epithelium originated solid tumour cell, any non-epithelium originated tumour cell, circulating vascular endothelial cell and tumour stem cell. The non-hematogenous karyocyte which from all epithelium originated solid tumour cell of humans, including but not limited to pancreatic cancer, lung cancer, cervical cancer, esophagus cancer, colorectal cancer, breast cancer, stomach cancer, liver cancer and so on; the non-epithelium originated tumour cell, e.g. melanoma.

Compared with existing general commercial red blood cell lysate on the market, the cell separation medium of the present invention is for human or patient only, and is capable of rapid separation of red blood cell without influencing all cell shape and cell characteristic, and stably improving the recovery rate of targeted cell from the original 60±20% to more than 95%. The cell separation medium is combined with a method using a reagent for removing leukocyte and a synchronous immunization-FISH method to realize efficient and rapid enrichment and extraction, and accurate identification of non-hematogenous karyocyte, e.g. tumour epithelial cell etc. in the body fluid of human or animal without an experiment step of breaking red blood cell, thereby having huge practical and potential clinical significance in aspects including early diagnosis of tumour, judgement of malignant hydrothorax and abdominal dropsy, udgement of neural tumour cell (e.g. glioma) in cerebrospinal fluid, and research and development of new anti-tumour drug and new drug for treating cardiovascular diseases etc.

### DETAILED DESCRIPTION OF THE EMBODIMENTS

The present invention provides a method for rapid and efficient separation of red blood cell in body fluid of human from malignant epithelial cell and other karyocyte by using a density separating agent (i.e. cell separation medium) on one hand. On the other hand, the present invention further provides a method of uniquely using a synchronous immunization-FISH technology for rapid and accurate identification of non-hematogenous karyocyte, e.g. tumour cell enriched from body fluid of human and animal, wherein the method combines immunofluorescent staining of surface antigen against human or animal leukocyte with FISH to implement synchronous staining of non-hematogenous karyocyte.

In a specific embodiment, the specific gravity of the cell separation medium at 20□ is 1.07156 to 1.07738 gr/ml; the cell separation medium includes any one or two or more components selected from the following reagents: polyvinylpyrrolidone-coated colloidal silica; polysaccharide; sodium diatrizoate or a derivative thereof; a nonionic polymer consisting of sucrose and epichlorohydrin; or any sugar-containing solution, e.g. dextran or sucrose; an iodinated small molecule compound, e.g. metrizamide, or any protein solution. Preferably, the specific gravity of the cell separation medium at 20□ is 1.07224 to 1.07568 gr/ml.

In a preferred embodiment, the monoclonal antibody used in the immunofluorescent staining specifically identify antigen of one or more of the following surface markers of human or animal leukocyte: CD3, CD31, CD34, CD45, CD50, CD69, CD84 or CD102.

A fluorescence labelling-probe or a probe combined group for the FISH of chromosome may specifically identify any one or more human or animal chromosomes.

In a preferred embodiment, the non-hematogenous karyocyte in the body fluid are selected from: any epithelium originated solid tumour cell (malignant epithelial cell), any non-epithelium originated tumour cell, circulating vascular endothelial cell and tumour stem cell in the body fluid of human or animal.

In a preferred embodiment, the non-hematogenous karyocyte is selected from epithelium originated solid tumour cell, e.g. lung cancer, cervical cancer, esophagus cancer, colorectal cancer, breast cancer, stomach cancer, liver cancer and so on; or selected from non-epithelium originated solid tumour cell, e.g. melanoma etc.

In a preferred embodiment, the non-hematogenous karyocyte (rare) in the body fluid is selected from one or more of the following cells: all epithelium originated solid tumour cell in the body fluid, any non-epithelium originated solid tumour cell, circulating vascular endothelial cell, tumour stem cell, stem cells and immune cell.

### Explanation of Nouns

Hematogenous karyocyte in human body fluid: referring to leukocyte, granulocyte, eosinophilic granulocyte, and basophilic granulocyte etc, wherein the leukocytes includes lymphocytes (T cells, B cells etc.).

Body fluid: including but not limited to hydrothorax or pleural effusion, abdominal dropsy or ascites, cerebrospinal fluid, lymph and/or blood etc.

Non-hematogenous karyocyte in body fluid of human or animal: mainly referring to rare cell including epithelium originated solid tumour cell or non-epithelium originated tumour cell and circulating vascular endothelial cell in the body fluid; tumour stem cell, stem cell, fetal cell in blood and some immune cell etc.

The present invention is mainly applied to rapid and efficient separation of red blood cell from malignant epithelial cell and other karyocyte in body fluid of human or animal. In addition, a synchronous immunization-FISH method combines immunofluorescent staining of surface antigen against human or animal leukocyte with FISH, thereby rapidly and accurately identifying non-hematogenous karyocyte, e.g. tumour cell etc. enriched from the body fluid.

A method for enrichment of non-hematogenous karyocyte from body fluid sample of the present invention includes the following major steps:
performing centrifugation to collect or remove plasma proteins;
placing the blood at the top layer of a cell separation medium and performing centrifugation;
taking the supernatant and adding immunomagnetic beads against coupling of leukocyte antibody to perform incubation and then adsorbing the immunomagnetic beads;
performing centrifugation and taking enriched cell precipitate;
smearing the enriched cell precipitate for subsequent identification.

A method for identification of non-hematogenous karyocyte, e.g. tumour cell enriched from the body fluid of the present invention includes:
performing FISH and staining using antibody against human or animal leukocyte synchronously,

Wherein the antibody refer to a CD antibody or combination of any CDs of human or animal, the CD as follows: CD3, CD31, CD34, CD45, CD50, CD69, CD84 or CD102.

In a composition of the present invention, the specific gravity of the cell separation medium at 20□ is 1.07156 to 1.07738 gr/ml or 1.07156 to 1.07738 gr/cm³; the cell separation medium includes any one or two or more components selected from the following reagents: polyvinylpyrrolidone-coated colloidal silica; polysaccharide; sodium diatrizoate or a derivative thereof; a nonionic polymer consisting of sucrose and epichlorohydrin; or any sugar-containing solution, e.g. dextran or sucrose; an iodinated small molecule compound, e.g. metrizamide, or any protein solution. The specific gravity of the cell separation medium may be prepared by any buffer solution having an osmotic pressure in the range of 260 to 320 mOsm/kg H₂O, and a pH value of 6.8 to 7.8.

### EXAMPLE

Rapid enrichment and identification of malignant non-hematogenous epithelial cell from the body fluid (blood or pleural effusion etc.) of patient by using the present invention.

7.5 ml of collected anticoagulation blood was centrifuged at room temperature for 5 minutes (700×g), plasma was removed to obtain blood, then the blood was added to the top layer of 10 ml of a cell separation medium, centrifuged was performed at room temperature for 5 minutes (500×g) to separate red blood cell. karyocyte at the upper layer was collected, 0.5 ml of immunomagnetic beads coated with monoclonal antibodies of human leukocyte surface antigen was added to the karyocytes, then incubation was performed for 60 minutes at room temperature. Then the magnetic beads were removed with a magnetic bracket. Then the supernatant was collected and centrifuged at room temperature for 3 minutes (1200×g) to obtain precipitated cell. Or (alternatively), the supernatant was collected and placed at the top layer of a cell separation medium and centrifuged over again, then the supernatant was collected and centrifuged at room temperature for 3 minutes (1200×g) to obtain precipitated cell, i.e. enriched non-hematogenous karyocyte, and the precipitated cell was smeared. CEP-7 and CEP-8 human chromosome probe+anti-human leukocyte antibody are applied to synchronous staining in subsequent identification experiments. Cells having an abnormal FISH signal and unstained by anti-human leukocyte antibody may be determined to be malignant non-hematogenous tumour cell in the body fluid.

Specifically, the specific gravity of the cell separation medium at 20° C. is preferably 1.07260 to 1.07650 gr/ml. The density in this specific range is applicable to separation of almost all karyocytes from red blood cell or immune microspheres or immune particles.

The cell separation medium includes any one or two or more of the following reagent components: polyvinylpyrrolidone-coated colloidal silica; polysaccharide; sodium diatrizoate or a derivative thereof; a nonionic polymer consisting of sucrose and epichlorohydrin; or any sugar-containing solution, e.g. dextran or sucrose; an iodinated small molecule compound, e.g. metrizamide, or any protein solution; the specific gravity of the cell separation medium may be regulated in the range of 1.07260 to 1.07650 gr/ml by any buffer solution having an osmotic pressure in the range of 285 to 315 mOsm/kg H₂O, and a pH value of 6.8 to 7.8 at 20° C. The specific gravity of the immune microspheres or immune particles is higher than that of the cell separation medium. The centrifugation based on the cell separation medium is performed in a common commercial centrifuge tube.

In the present invention, monoclonal antibody used in staining of antibody against human or animal leukocyte may specifically identify one or more of the following leukocytes surface markers, but are not limited thereby: CD3, CD31, CD34, CD45, CD50, CD69, CD84 or CD102 etc.

It would be understood by the person skilled in the art that the above preferred examples are only to specifically describe the present invention but not to limit the present invention. Various improvements, combinations, sub-combinations and alterations thereto can be made as needed. All improvements, combinations, sub-combinations, alterations and equivalent substitutions fall into the scope of the appended claims.

1. A method for identification of non-hematogenous karyocyte enriched from body fluid of human or animal, wherein immunofluorescent staining of antibody against human or animal leukocyte and Fluorescence In Situ Hybridization of chromosome are carried out synchronously to distinguish hematogenous karyocyte.

2. The method according to claim 1, wherein the antibody against human or animal leukocyte in the immunofluorescent staining specifically identify antigen of one or more of the following surface markers of the human or animal leukocyte: CD3, CD31, CD34, CD45, CD50, CD69, CD84 or CD102.

3. The method according to claim 1, wherein a fluorescence labelling-probe or a probe combined group for the FISH of chromosome may specifically identify any one or more human or animal chromosomes.

4. A method for efficient and rapid separation of red blood cell and non-hematogenous karyocyte in body fluid of human, wherein the method does not need a step of lysing and breaking red blood cell wherein a cell separation medium is adopted in the separation; the specific gravity of the cell separation medium at 20° C. is 1.07156 to 1.07738 gr/ml; the cell separation medium comprises any one or two or more components selected from the following reagents: polyvinylpyrrolidone-coated colloidal silica; sodium diatrizoate or a derivative thereof; a nonionic polymer consisting of sucrose and epichlorohydrin; or any sugar-containing solution, e.g. dextran, sucrose or polysaccharide; an iodinated small molecule compound, e.g. metrizamide, or any protein solution.

5. A method for enrichment of non-hematogenous karyocyte from body fluid of human, wherein the method does not need a step of lysing and breaking red blood cell, and the method comprises the following steps:
performing centrifugation to collect or remove plasma protein;
placing the centrifuged supernatant at the top layer of a cell separation medium and performing centrifugation over again;
taking the centrifuged supernatant and adding immunomagnetic beads against coupling of leukocyte antibody to perform incubation and then adsorbing the immunomagnetic beads;
placing the incubated supernatant at the top layer of the cell separation medium and performing centrifugation over again;
performing centrifugation for the centrifuged supernatant to enrich cell precipitate.

6. A method for enrichment and identification of non-hematogenous karyocyte from body fluid of human or animal, wherein it comprises:
performing centrifugation to remove plasma protein;
placing the centrifuged supernatant at the top layer of a cell separation medium and performing centrifugation over again;
taking the centrifuged supernatant and adding immunomagnetic beads against coupling of leukocyte antibody to perform incubation and then adsorbing the immunomagnetic beads;
placing the incubated supernatant at the top layer of the cell separation medium and performing centrifugation over again;
taking and performing centrifugation for the supernatant and taking enriched cell precipitate,
wherein immunofluorescent staining of antibody against human or animal leukocyte and FISH of marker against human or animal tumour cell or epithelial cell marker and chromosome are carried out synchronously to distinguish non-hematogenous tumour cell.

7. The method according to claim 6, wherein the body fluid of human or animal comprises hydrothorax or pleural effusion, abdominal dropsy or ascites, cerebrospinal fluid, lymph and/or blood, urine, sputum, lavage fluid and so on.

8. The method according to claim 6, wherein the non-hematogenous karyocyte in the body fluid of human or animal are selected from a group consisting of the followings: any epithelium originated solid tumour cell, any non-epithelium originated tumour cell, circulating vascular endothelial cell and tumour stem cell.

9. The method according to claim 6, wherein the non-hematogenous karyocyte is from all epithelium originated solid tumour cell of human or animal, comprising pancreatic cancer, lung cancer, cervical cancer, esophagus cancer, colorectal cancer, breast cancer, stomach cancer, liver cancer and so on; from the non-epithelium originated tumour cell comprising melanoma.

10. The method according to claim 6, wherein the non-hematogenous karyocyte comprises tumour cell and vascular endothelial cell.
